(19) United States
(12) Patent Application Publication
(10) Pub. No.: US 2007/0259376 A1
(43) Pub. Date: Nov. 8, 2007

(54) Homocysteine immunoassay

(76) Inventor: Toru Yoshimura, Matsudo-shi (JP)

Correspondence Address: ROBERT DEBERARDINE;ABBOTT LABORATORIES, 100 ABBOTT PARK ROAD, DEPT. 377/AP6A, ABBOTT PARK, IL, 60064-6008, US

(21) Appl. No.: 11/646,697

(22) Filed: Dec. 28, 2006

Related U.S. Application Data
(63) Continuation-in-part of application No. PCT/US06/49024, filed on Dec. 21, 2006.

(30) Foreign Application Priority Data
Dec. 22, 2005 (JP) 2005-369198

(51) Int. Cl. G01N 33/53

(52) U.S. Cl. 435/7.1

(57) ABSTRACT

In immunoassays such as homocysteine immunoassays, it is known that the measured values can be dispersed due to the influence of inhibiting substances present in a biological sample on the immunoreaction. The present invention provides among other things a method whereby the influence of the immunoreaction inhibiting substances is easily and efficiently suppressed or eliminated, and also provides a kit used for such a method. The influence of the immunoreaction inhibiting substances is suppressed or eliminated when the analyte (e.g., homocysteine) contained in the biological sample is reacted with the antibody in the presence of extrinsic polyanion.

### RELATED APPLICATIONS

The application is a continuing application (continuation-in-part) under 35 U.S.C. Section 111(a) of PCT International Application PCT/US__/______ filed Dec. 21, 2006 (entitled “Homocysteine Immunoassay” and filed as Express Mail EV959282592US, Docket No. 8529WO01), pending, which claims the benefit of Japanese Patent Application 2005-369198 filed Dec. 22, 2005.

### FIELD OF THE INVENTION

The present invention relates generally to an improved immunoassay that includes polyanion. Among other things the invention relates to a system for the detection of analyte (e.g., homocysteine) using an improved immunoassay. In particular, the invention relates among other things to diagnostic tests, methods of use, and kits related to the assessment of analyte (e.g., homocysteine) levels in a biological sample. Optionally in such an immunoassay the influence of inhibiting substances in the biological sample is eliminated by inclusion in the assay of polyanion.

### BACKGROUND OF THE INVENTION

The recognition in recent years of high values of plasma homocysteine as being one of the dangerous factors for cardiovascular disease has resulted in an increased awareness in the clinical field of the necessity of measurement of homocysteine levels in serum or plasma.

One method for the measurement of homocysteine using an immunological means has been developed by Erling et al. (Japanese Patent Laid-Open No. 05/513,023). In this competitive immunoassay the homocysteine concentration is measured after a pretreatment step comprising a first stage where homocysteine bound to components in blood by a disulfide bond is dissociated, and a second stage where the dissociated homocysteine is reacted with an enzyme (S-adenosylhomocysteine hydrolase) together with an auxiliary substrate (adenosine) so as to convert the homocysteine into a form capable of being measured by immunoassay.

Other options for measuring the concentration of homocysteine include separation and measurement conducted based on physical and chemical properties of homocysteine using high performance liquid chromatography (HPLC) (Journal of Chromatography B, volume 779, number 2, Nov. 5, 2002, pages 359 to 363) or mass spectral analysis (MS). These methods are complicated and not easily adapted to testing of large numbers of clinical samples.

Thus, to date there has been development of a measuring method for homocysteine that is superior to an immunoassay in view of simplicity and convenience. However, when serum or plasma samples are measured by conventional immunoassays for homocysteine, error of the measurement results. This is believed to be due to the influence of inhibiting substances present in blood.

In order to eliminate the influence of such inhibiting substances, a treatment method has been developed for use in conventional and automated immunoassays. In this method the biological sample is directly diluted or the volume of the reaction solution is increased. Such a treatment method is disadvantageous in that it requires increased labor for the measurement of homocysteine and results in an unnecessary decrease in sensitivity due to the dilution. Moreover, in the measurement by a fully automated measuring apparatus, the dilution step causes a decrease in the speed of sample processing as well as an inability of some of the measuring apparatuses to handle the increased volume of the reaction solution.

In other assays and systems separate and apart from a homocysteine immunoassay, polyanions such as heparin have been employed for a variety of different reasons. In Japanese Patent Laid-Open No. 08/145,998, Mori et al. added heparin in an immunoassay of insulin-like growth factor to inhibit the recombination of insulin-like growth factor with insulin after treatment of the biological sample liberating the factor from insulin. Baker and Ishikawa et al. use polyanion as a connecting substance for making antigen or antibody into a solid phase (Japanese Patent Laid-Open No. 07/507,871 and Japanese Patent Laid-Open No. 02/168,162). Yoshimura et al. use polyanion in a chromatographic assay device for the neutralization of polycation employed for the separation of red blood cells (Japanese Patent Laid-Open No. 2002/509,254). Kurokawa et al. teach that the influence of an interfering substance in an immunoassay using a complete antibody is eliminated by addition of heparin (Japanese Patent Laid-Open No. 08/029,420). And, in a chromatographic assay system using colloid particles, Sakamoto et al. report that the non-specific aggregation reaction is inhibited and the generation of false positives avoided by the addition of heparin to a blood sample (Japanese Patent Laid-Open No 07/151,754).

Based on the foregoing, there remains a need for the development of a method whereby the influence of inhibiting substances in an immunoassay of homocysteine is eliminated. Optimally such a method can be done without requiring increased labor or sample processing time, without substantially diluting the sample, and/or without deteriorating the sensitivity of the assay. Therefore, it is an object of the invention to provide among other things diagnostic tests, methods of use, and kits for the assessment of homocysteine levels in a biological sample, optionally making use of a polyanion. Optimally the tests, methods and kits of the invention avoid some of the pitfalls in homocysteine testing which are inherent in the currently used methodologies. These and other objects will be apparent from the description provided herein.

The foregoing discussion of background information is provided merely to assist the reader in understanding the invention and is not admitted to describe or constitute prior art to the invention.

### SUMMARY OF THE INVENTION

Among other things the present description provides an improvement of a immunoassay of a biological sample (e.g., an assay of an analyte such as a factor for cardiovascular disease, including but not limited to a homocysteine immunoassay), characterized in that the sample is reacted with antibody in the presence of an extrinsic polyanion. Optionally the polyanion is selected from the group consisting of heparin, polyacrylic acid, and dextran sulfate. In one embodiment as described herein, the polyanion concentration ranges from about 1 μg/mL to about 100 mg/mL.

Optionally the method of the invention can be employed for an immunoassay (e.g., an assay of an analyte, such as a factor for cardiovascular disease, including but not limited to a homocysteine immunoassay) using any sort of appropriate immunoreaction carried out on any appropriate instrument. In one embodiment the immunoassay comprises a competitive immunoassay. In another embodiment the immunoassay is carried out using an automated measuring apparatus. In yet another embodiment, the immunoassay comprises a sandwich assay.

The invention thus provides a method for assaying a biological sample for an analyte of interest (e.g., a cardiovascular antigen such as homocysteine), optionally wherein the method comprises:

(a) obtaining a biological sample from a subject (e.g., from a human subject);

(b) reacting the biological sample with antibody specific for analyte (e.g., with antibody that reacts with homocysteine) in the presence of polyanion;

(c) detecting the binding of analyte (e.g., homocysteine) present in the sample with said antibody by any appropriate means; and

(d) quantifying the binding as a measure of the amount of the analyte (e.g., homocysteine) present in the sample. Optimally the reaction of the antibody with analyte in the presence of polyanion is done where the polyanion is either added before, during, or after the reaction of the antibody with analyte. In one embodiment, polyanion is added either before or during the reaction of the antibody with analyte.

Also provided by the description herein are kits to be used for the immunoassay according to by the invention (e.g., an assay of a factor for cardiovascular disease, including a homocysteine immunoassay), wherein the kit comprises a polyanion.

These and other features, aspects, objects, and embodiments of the invention will become more apparent in the following detailed description (including the drawings) which contains information on exemplary features, aspects, objects and embodiments of the invention.

### BRIEF DESCRIPTION OF THE DRAWINGS

FIG. 1 is a graph of changes in signal intensity caused by addition of the polyanion heparin in a measuring system for homocysteine, as described in Example 1. The ordinate shows the measured signal whereas the abscissa shows the concentration of the added heparin in the reaction (μg/mL). Symbols: -▪-, serum being measured in an undiluted state; -●-, serum being measured in high concentration; -▾-, plasma in blood collected with heparin.

FIG. 2 is a graph of the ratio of the concentration of homocysteine in the absence of the polyanion heparin as compared to the known value measured using a chemiluminescence automated measuring apparatus, as described in Example 2. The ordinate shows the measured values for known values (%) whereas the abscissa shows known homocysteine concentrations (μM).

FIG. 3 is a graph of the ratio of the concentration of homocysteine in the presence of the polyanion heparin as compared to the known value measured using a chemiluminescence automated measuring apparatus, as described in Example 2. The ordinate shows measured values for known values (%) whereas the abscissa shows known homocysteine concentrations (μM).

FIG. 4 is a bar chart showing the influence of polyanion on the dissociation rate where the apparent homocysteine concentration is from a “high” concentration sample group and a “normal” concentration sample group, as described in Example 3. The ordinate shows dissociation rate whereas the abscissa shows the amount of polyanion added. Bars (left to right): (a) no polyanion added; (b) 4.2 μg/mL of heparin; (c) 14 μg/mL of heparin; (d) 42 μg/mL of heparin; (e) 84 μg/mL of heparin; (f) 420 μg/mL of heparin; (g) 420 μg/mL of dextran sulfate; (h) 42 μg/mL of polyacrylic acid; (i) 420 μg/mL of polyacrylic acid; (j) 2.8 mg/mL of gelatin; and (k) 281 μg/mL of bovine γ-globulin.

### DETAILED DESCRIPTION OF THE INVENTION

The present description relates to a method for improving an immunoassay by the addition of a polyanion. Not willing to be bound by any theory, after intensive investigation as described herein, it surprisingly has been discovered that a substance which is the same as or similar to polyanion in terms of either structure of function appears to be causing inhibition of the immunoreaction in a homocysteine immunoassay, and that variations in the amount of this polyanion-like substance contained in a sample result in dispersion in immunoassayed values of homocysteine. Because it typically is not easy to remove a specific substance existing in blood without deleteriously impacting assay results, the description herein provides a method and means for substantially reducing or eliminating the influence of the immunoreaction inhibiting substance by adding to the immunoassay a sufficient amount of a polyanion.

The present invention thus provides, among other things, diagnostic tests, methods of use, and kits for the assessment of a cardiovascular factor such as homocysteine. These and additional embodiments, features, aspects, illustrations, and examples of the invention are further described in the sections which follow.

Definitions

Unless defined otherwise herein, all technical and scientific terms used herein have the same meaning as commonly understood by one of ordinary skill in the art to which this invention belongs.

“Samples” or “biological samples” that can be assayed using the methods of the present invention include biological fluids, such as whole blood, serum, plasma, synovial fluid, cerebrospinal fluid, bronchial lavage, ascites fluid, bone marrow aspirate, pleural effusion, urine, as well as tumor tissue or any other bodily constituent or any tissue culture supernatant that could contain the analyte of interest. Preferred biological samples in an immunoassay of homocysteine are further described below.

“Analyte,” as used herein, refers to the substance to be detected, which may be present in the sample (i.e., the biological sample). The analyte can be any substance for which there exists a naturally occurring specific binding partner or for which a specific binding partner can be prepared. Thus, an analyte is a substance that can bind to one or more specific binding partners in an immunoassay. One example of an analyte as described herein is an endogenous antigen and/or cardiovascular factor, including but not limited to homocysteine. A cardiovascular factor typically is an endogenous factor such as an antigen that can be assessed as a measure of, or measure of risk of developing, cardiovascular disease.

A “binding partner,” as used herein, is a member of a binding pair, i.e., a pair of molecules wherein one of the molecules binds to the second molecule. Binding partners that bind specifically are termed “specific binding partners.” In addition to the antigen and antibody binding partners commonly used in immunoassays, other specific binding partners can include biotin and avidin, carbohydrates and lectins, complementary nucleotide sequences, effector and receptor molecules, cofactors and enzymes, enzyme inhibitors and enzymes, and the like. Furthermore, specific binding partners can include partner(s) that is/are analog(s) of the original specific binding partner, for example, an analyte-analog. Immunoreactive specific binding partners include antigens, antigen fragments, antibodies and antibody fragments, both monoclonal and polyclonal, and complexes thereof, including those formed by recombinant DNA methods.

As used herein, the term “epitope”, “epitopes” or “epitopes of interest” refer to a site(s) on any molecule that is recognized and is capable of binding to a complementary site(s) on its specific binding partner. The molecule and specific binding partner are part of a specific binding pair. For example, an epitope can be a polypeptide, protein, hapten, carbohydrate antigen (such as, but not limited to, glycolipids, glycoproteins or lipopolysaccharides) or polysaccharide and its specific binding partner, can be, but is not limited to, an antibody, e.g., an autoantibody. Typically an epitope is contained within a larger antigenic fragment (i.e., region or fragment capable of binding an antibody) and refers to the precise residues known to contact the specific binding partner. It is possible for an antigenic fragment to contain more than one epitope.

As used herein, “specific” or “specificity” in the context of an interaction between members of a specific binding pair (e.g., an antigen and antibody) refers to the selective reactivity of the interaction. The phrase “specifically binds to” and analogous terms thereof refer to the ability of antibodies to specifically bind to an analyte (e.g., an endogeneous antigen such as homocysteine) and not specifically bind to other entities. Antibodies or antibody fragments that specifically bind to an analyte can be identified, for example, by diagnostic immunoassays (e.g., radioimmunoassays (“RIA”) and enzyme-linked immunosorbent assays (“ELISAs”) (See, for example, Paul, ed., Fundamental Immunology, 2nd ed., Raven Press, New York, pages 332-336 (1989)), BIAcore® (Sweden), KinExA® (Kinetic Exclusion Assay, available from Sapidyne Instruments (Boise, Id.)) or other techniques known to those of skill in the art. The term “specifically binds” indicates that the binding preference (e.g., affinity) for the target molecule/sequence is at least 2-fold, more preferably at least 5-fold, and most preferably at least 10- or 20-fold over a non-specific target molecule (e.g. a randomly generated molecule lacking the specifically recognized site(s)).

A “solid phase,” as used herein, refers to any material that is insoluble, or can be made insoluble by a subsequent reaction. The solid phase can be chosen for its intrinsic ability to attract and immobilize a capture agent. Alternatively, the solid phase can have affixed thereto a linking agent that has the ability to attract and immobilize the capture agent. The linking agent can, for example, include a charged substance that is oppositely charged with respect to the capture agent itself or to a charged substance conjugated to the capture agent. In general, the linking agent can be any binding partner (preferably specific) that is immobilized on (attached to) the solid phase and that has the ability to immobilize the capture agent through a binding reaction. The linking agent enables the indirect binding of the capture agent to a solid phase material before the performance of the assay or during the performance of the assay. The solid phase can, for example, be plastic, derivatized plastic, magnetic or non-magnetic metal, glass or silicon, including, for example, a test tube, microtiter well, sheet, bead, microparticle, chip, and other configurations known to those of ordinary skill in the art.

As used herein, term “microparticle” refers to a small particle that is recoverable by ultracentrifugation. Microparticles typically have an average diameter on the order of about 1 micron or less.

The term “capture agent” is used herein to refer to a binding partner that binds to analyte, preferably specifically. Capture agents can be attached to a solid phase. As used herein, the binding of a solid phase-affixed capture agent to analyte forms a “solid phase-affixed complex.”

The term “labeled detection agent” is used herein to refer to a binding partner that binds to analyte, preferably specifically, and is labeled with a detectable label or becomes labeled with a detectable label during use in an assay.

A “detectable label” includes a moiety that is detectable or that can be rendered detectable.

As used with reference to a labeled detection agent, a “direct label” is a detectable label that is attached, by any means, to the detection agent.

As used with reference to a labeled detection agent, an “indirect label” is a detectable label that specifically binds the detection agent. Thus, an indirect label includes a moiety that is the specific binding partner of a moiety of the detection agent. Biotin and avidin are examples of such moieties that are employed, for example, by contacting a biotinylated antibody with labeled avidin to produce an indirectly labeled antibody.

As used herein, the term “indicator reagent” refers to any agent that is contacted with a label to produce a detectable signal. Thus, for example, in conventional enzyme labeling, an antibody labeled with an enzyme can be contacted with a substrate (the indicator reagent) to produce a detectable signal, such as a colored reaction product.

As used herein, an “antibody” refers to a protein consisting of one or more polypeptides substantially encoded by immunoglobulin genes or fragments of immunoglobulin genes. This term encompasses polyclonal antibodies, monoclonal antibodies, and fragments thereof, as well as molecules engineered from immunoglobulin gene sequences. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

A typical immunoglobulin (antibody) structural unit is known to comprise a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms “variable light chain (VL)” and “variable heavy chain (VH)” refer to these light and heavy chains respectively.

Antibodies exist as intact immunoglobulins or as a number of well-characterized fragments produced by digestion with various peptidases. Thus, for example, pepsin digests an antibody below the disulfide linkages in the hinge region to produce F(ab′)2, a dimer of Fab which itself is a light chain joined to VH-CH1 by a disulfide bond. The F(ab′)2 may be reduced under mild conditions to break the disulfide linkage in the hinge region thereby converting the (Fab′)2 dimer into a Fab′ monomer. The Fab′ monomer is essentially a Fab with part of the hinge region (see, Fundamental Immunology, W. E. Paul, ed., Raven Press, N.Y. (1993), for a more detailed description of other antibody fragments). While various antibody fragments are defined in terms of the digestion of an intact antibody, one of skill will appreciate that such Fab′ fragments may be synthesized de novo either chemically or by utilizing recombinant DNA methodology.

Thus, the term “antibody,” as used herein also includes antibody fragments either produced by the modification of whole antibodies or synthesized de novo using recombinant DNA methodologies. Preferred antibodies include single chain antibodies (antibodies that exist as a single polypeptide chain), more preferably single chain Fv antibodies (sFv or scFv), in which a variable heavy and a variable light chain are joined together (directly or through a peptide linker) to form a continuous polypeptide. The single chain Fv antibody is a covalently linked VH-VL heterodimer which may be expressed from a nucleic acid including VH- and VL-encoding sequences either joined directly or joined by a peptide-encoding linker. Huston, et al. (1988) Proc. Nat. Acad. Sci. USA, 85: 5879-5883. While the VH and VL are connected to each as a single polypeptide chain, the VH and VL domains associate non-covalently. The scFv antibodies and a number of other structures converting the naturally aggregated, but chemically separated, light and heavy polypeptide chains from an antibody V region into a molecule that folds into a three dimensional structure substantially similar to the structure of an antigen-binding site are known to those of skill in the art (see, e.g., U.S. Pat. Nos. 5,091,513, 5,132,405, and 4,956,778).

As used herein, the singular forms “a”, “an” and “the” include plural references unless the context clearly dictates otherwise.

As used herein, the term “about” refers to approximately a +/−10% variation from the stated value. It is to be understood that such a variation is always included in any given value provided herein, whether or not it is specifically referred to.

Immunoassay

Thus the present invention provides among other things an immunoassay of a biological sample, including an immunoassay of a cardiovascular factor, e.g., homocysteine, present in the sample. The method comprises inclusion of a polyanion in the immunoassay (e.g., a homocysteine immunoassay) in the reaction of the biological sample with an antibody. When the immunoassay of the present invention is used, it is possible to reduce or eliminate the influence of inhibiting substances existing in a sample without substantial dilution of the sample, or without using a large amount of an assay buffer solution, at the same time giving a highly sensitive and highly reliable measured value of homocysteine in a simple and convenient manner. This is particularly advantageous where the immunoassay (e.g., the homocysteine immunoassay) is fully automated and a large amount of sample is to be treated within a short time.

The description provided herein pertains among other things to a homocysteine immunoassay. However, it is expected that the methods described herein can be more generally applied, and that the inclusion of exogenous polyanion in the reacting step of a biological sample with an antibody against the antigen of interest would reduce variance in measurement of other analytes, particularly other cardiovascular factors (e.g., endogenous cardiovascular antigens) in biological samples.

The immunoassay employed in the method of the present invention can be any immunoassay so long as it is a detection (e.g., quantitative) method that utilizes an antigen-antibody reaction. Accordingly, it can be any of the methods routinely employed for immunoassay including but not limited to a competitive method and a non-competitive method such as a sandwich method.

Biological Sample Collection and Processing

The assay methods of the invention are generally carried out on samples derived from an animal, preferably a mammal, and more preferably a human. These methods can be carried out on samples from asymptomatic subjects or subjects with one or more symptoms of disease.

The methods of the invention can be carried out using any sample that may contain analyte of interest, e.g., that may contain homocysteine. Convenient samples include, for example, blood, serum, and plasma. The biological sample which is an object for the measurement of homocysteine in the immunoassay of the present invention can be any biological sample so long as it is a liquid sample derived from a living organism such as a body fluid or a tissue extract and, usually, it is preferred to use serum, plasma or urine.

The sample may be pretreated, as necessary or desired, by dilution in an appropriate buffer solution or other solution, or optionally may be concentrated. Any of a number of standard aqueous buffer solutions, employing any of a variety of buffers, such as phosphate, Tris, or the like, optionally at physiological pH, can be used.

In regards to a homocysteine immunoassay in particular, since many homocysteines bind to other thiol or protein such as albumin by means of a disulfide bond in a biological sample, it is typical for the measurement of total homocysteine in plasma, urine, and other samples to subject the sample to pretreatment with a reducing agent such as dithiothreitol (DTT).

Because no antibody which selectively recognizes homocysteine (e.g., as present in untreated biological sample) is yet available, it further is typical in an immunoassay for homocysteine that the homocysteine is subjected to an enzymatic treatment or the like to be converted into a molecule which can be recognized by antibody. The enzyme and any auxiliary substrate used in such a pretreatment step can be any appropriate reagents so long as they are enzyme and auxiliary substrate which are able to convert homocysteine into a molecule which is can be measured immunologically. For example, it is common to use S-adenosylhomocysteine hydrolase as an enzyme and adenosine as an auxiliary substrate. In that case, homocysteine is converted to S-adenosylhomocysteine and is subjected to an immunoassay.

An auxiliary substrate such as adenosine preferably is charged in a solution of a reducing agent, but optionally ca be added to any reagent so long as it is a reagent which can be added to a pretreated solution or during the first reaction without deleteriously impacting the reaction.

Polyanion

A polyanion is “exogenous” in the sense that it typically is added to an immunoreaction, as described herein. Although the optimum amount of the polyanion used according to the present invention varies depending upon the type of the polyanion employed, optionally not less than about 1 μg/mL of polyanion is added during the reaction with a sample. Typically the serum amount in the reaction solution is from about 6% to about 20%, optionally about 10%. Use of not less than about 1 μg/mL of polyanion typically provides that the influence of the inhibiting substances is substantially reduced, if not completely eliminated. Regardless of the magnitude of impact, however, use of not less than about 1 μg/mL of polyanion in a homocysteine immunoassay will allow an effect of the addition to be observed. In some circumstances, it may be desirable to include a lesser amount of polyanion in the homocysteine assay. For instance, use of too much polyanion could result in an increase in manufacturing cost and also cause difficulties in terms of mechanical operation (e.g., such as insufficient dispensing amount due to an increase in viscosity). Although the upper limit of the amount used varies depending upon the type of polyanion employed, optionally it is no more than about 100 mg/mL. Although it depends upon the type of polyanion and the amount of serum used for the immunoassay, the optimum polyanion concentration is, therefore, within a range of from about 1 μg/mL to about 100 mg/mL (e.g., from about 1 μg/mL to about 100 μg/mL, from about 100 μg/mL to about 100 mg/mL, or from about 50 μg/mL to about 50 mg/mL) during the reaction of the antibody with the biological sample, in an immunoassay in which the serum or plasma amount in the reaction solution is about 10%. There is a tendency that, when the sample amount is a little, the effect is noted with less concentration, whereas when the sample amount is high, a significant effect is noted by higher concentration.

For the exogenous (added) polyanion to achieve a desired effect in the present invention, it is necessary that the polyanion coexists during the reaction of homocysteine in the sample with the antibody. Any route may be employed for addition of the polyanion. Thus, polyanion may be added to any reagent so long as it is a reagent which participates and does not interfere with the reaction of homocysteine with antibody. For example, exogenous polyanion may be added to any solution such as solid-phase antibody solution, labeled solution, assay buffer solution, pretreatment solution, and the like. Exemplary solutions include but are not limited to: Tris buffer; phosphate buffer; borate buffer; Good's buffer; SSC buffer; TBE buffer; TAE buffer; and any buffer that is routinely employed in an immunoassay.

In the present description, a “polyanion” is a molecule in which the anion is present in multivalent form, i.e., is in more in one molecule such that there is a valence of three or more, and optionally is tetravalent. Where the anion existing in one molecule is more than decavalent (i.e., has a valence greater than ten) and the molecular weight is several hundred or more, the molecule shows a significant property as polyanion. Optimally the upper limit of molecular weight of a polyanion employed as described herein is set within limits such that the viscosity does not deleteriously impact the measuring system (i.e., immunoassay). Typically, a molecular weight of several hundred thousand to several million is the upper limit for the polyanion.

With regard to the polyanion used in the present invention, any type may be used so long as it meets the aforementioned definition. Examples of suitable polyanions include but are not limited to: polyacrylic acid where the carboxyl group is present in a multivalent state in a polymer chain of carbon, and substances similar thereto; dextran sulfate where the polysaccharide is substituted with a sulfate group; heparin; heparin sulfate; poly(methyl methacrylate) (PMMA); poly(vinylsulfonic acid) (PVSA); poly-L-aspartic acid; and carboxymethyl cellulose (CMC). Other polyanions that optionally can be employed include but are not limited to chondroitan sulfate, hyaluronic acid, dermatan sulfate, and dextran sulfate. Such a polyanion optionally can be used in the form of a salt, e.g., sodium salt, lithium salt, or other similar salt. The polyanion used can be a sole polyanion, or can be a mixture of different types of polyanions (e.g., a so-called “plurality” wherein the plurality optionally comprises between two and five, added either simultaneously or sequentially).

Antibody

The antibody used in the present invention preferably is an antibody that is able to recognize analyte of interest (e.g., homocysteine). Optionally the homocysteine is first subjected to a conversion treatment so as to render it capable of being recognized by an antibody. For example, when the sample is previously treated with S-adenosylhomocysteine hydrolase, an anti-S-adenosylhomocysteine antibody is used. Such an antibody can be any polyclonal antibody or monoclonal antibody. Moreover, the antibody can be not only a complete antibody but also any type of an antibody fragment including Fab, Fab′, and F(ab′)₂, or antibody fragment where only the active site is taken out by means of genetic recombination, so long the antibody provides a specific activity (i.e., a reactivity).

Scoring

The immunoassays according to the invention optimally are scored in accordance with standard practice and, optionally include the use of positive and/or negative controls and/or or standards (calibrators) containing known concentrations of antibodies to the analyte of interest. The level of analyte (e.g., homocysteine) optionally is compared with a control level or control range, which can be determined when the assay is carried out or, more conveniently, can be predetermined.

Other Reagents

With regard to the other reagents used in the immunoassay of the present invention (e.g., reagents such as antibody, labeled substance, reducing sugar and enzyme), substances which are routinely used in homocysteine immunoassays can likewise be employed in the assay as described herein according to their ordinary and customary conditions for use. This is further expanded upon below.

Immunoassay Methods—In General

The immunoassay methods of the invention can be carried out in any of a wide variety of formats. These formats merely are modified as described above to include polyanion in the reacting step of analyte antigen (e.g., homocysteine) with antibody. For a general review of immunoassays, see Methods in Cell Biology Volume 37: Antibodies in Cell Biology, Asai, ed. Academic Press, Inc. New York (1993); Basic and Clinical Immunology 7th Edition, Stites & Terr, eds. (1991), which is incorporated by reference in its entirety.

In particular embodiments, an immunoassay method of the invention can be performed by contacting a biological sample suspected of containing an analyte of interest, with an antibody reactive therewith in the presence of polyanion, and under conditions sufficient for binding of the antibody to any analyte present in the biological sample. Analyte is detected/quantitated by detecting complex(es) comprising the analyte antigen bound to the reactive antibody. Such assays can be homogeneous or heterogeneous (i.e., employing a solid phase). In heterogeneous assays, a capture agent that binds to the analyte is typically affixed to a solid phase. Analytes such as homocysteine can be measured in a non-competitive immunoassay, wherein the amount of analyte bound to antibody is positively correlated with the concentration of analyte present in the biological sample.

In other embodiments, the biological sample is contacted with the antibody reactive with analyte (and which may, but need not, be affixed to a solid phase) and also contacted with another antibody that reacts with analyte so as to form a “sandwich” where the analyte is bound between two antibody reagents. Analyte is detected/quantitated by detecting complex(es) comprising the antigen bound to the reactive antibodies.

For example, in one format of a sandwich immunoassay, an embodiment of the invention, the first antibody is affixed to a solid phase, binding of analyte antigen present in the biological sample to the antibody forms a solid phase-affixed complex, and detecting comprises detecting a signal from the solid phase-affixed complex. In particular embodiments of this format, the solid phase-affixed complex is detected using a second antibody also reactive with analyte antigen and that is directly or indirectly labeled. The bound entities are separated, if necessary, from free labeled antibody, typically by washing, and the signal from the bound label is detected.

Analyte (e.g., antigen such as homocysteine) can also be measured in competitive immunoassay, wherein the signal is negatively correlated with the concentration of analyte present in the biological sample. In an example of a competitive format, the biological sample is contacted with an antibody (which may, but need not, be affixed to a solid phase) and also is contacted with competing labeled (directly or indirectly) antigen. This step is carried out under conditions sufficient for specific binding of the labeled antigen and analyte antigen to the antibody. The labeled antigen and analyte antigen compete with each other for binding to the antibody. Accordingly, the higher the level of analyte antigen (such as homocysteine) in a biological sample, the lower is the binding of labeled antigen to the antibody. The biological sample may be contacted with the labeled antigen and the antibody either simultaneously or sequentially, in any order.

Competitive immunoassays of this type can be conveniently carried out using a solid phase-affixed antibody. In this case, binding of the analyte antigen present in the biological sample to antibody forms a solid phase-affixed complex, and detection entails detecting a signal from the solid phase-affixed complex. The bound entities are separated, if necessary, from free labeled antigen, typically by washing, and the signal from any bound label (displacing analyte antigen) is detected.

Capture Agent

Capture agents useful in the immunoassay methods of the invention include those that bind to analyte antigen (e.g., homocysteine) and can be affixed to a solid phase. Convenient capture agents include antibodies specific for the analyte antigen.

Analyte Antigens

Any endogenous antigen can be used (e.g., assessed as the analyte antigen or included in a kit as a calibrator or control) in the immunoassay methods of the invention.

In particular embodiments, the endogenous antigen is an endogenous antigen amino acid sequence that can be derived from any organism. Endogenous antigen amino acid sequences useful in the invention are generally those derived from vertebrates, preferably from birds or mammals, more preferably from animals having research or commercial value or value as pets, such as mice, rats, guinea pigs, rabbits, cats, dogs, chickens, pigs, sheep, goats, cows, horses, as well as monkeys and other primates. In particular embodiments, the endogenous antigen amino acid sequence is derived from a human polypeptide.

The methods of the invention can employ full-length endogenous antigens or one or more fragments thereof. Fragments will generally have at least one epitope to which an antibody can bind. Such fragments can have a length, e.g., of about 125, 100, 75, 50, 25, or 15 amino acids or a length that falls within a range with endpoints defined by any of these values (e.g., 15-125, 25-100, 50-75, 15-100, etc.).

The endogenous antigen amino acid sequence can be a wild-type amino acid sequence or an amino acid sequence variant of the corresponding region of a wild-type polypeptide. In certain embodiments, endogenous antigens include a wild-type endogenous antigen amino acid sequence or an endogenous antigen amino acid sequence containing conservative amino acid substitutions, as defined above.

Endogenous antigens useful in the invention can include other amino acid sequences, including those from heterologous proteins. Accordingly, the invention encompasses fusion polypeptides in which an endogenous antigen amino acid sequence is fused, at either or both ends, to amino acid sequence(s) from one or more heterologous proteins. Examples of additional amino acid sequences often incorporated into proteins of interest include a signal sequence, which facilitates purification of the protein, and an epitope tag, which can be used for immunological detection or affinity purification.

Endogenous antigen polypeptides according to the invention can be synthesized (e.g., for use as calibrators or controls in the kits according to the invention) using methods known in the art, such as for example exclusive solid phase synthesis, partial solid phase synthesis, fragment condensation, and classical solution synthesis. See, e.g., Merrifield, J. Am. Chem. Soc., 85:2149 (1963). For a description of solid phase peptide synthesis procedures, see John Morrow Stewart and Janis Dillaha Young, Solid Phase Peptide Syntheses (2nd Ed., Pierce Chemical Company, 1984).

Endogenous antigen polypeptides can also produced using recombinant techniques. In certain embodiments, the sequence of an endogenous antigen coding region is used as a guide to design a synthetic nucleic acid molecule encoding the endogenous antigen polypeptide that can be incorporated an expression vector. Methods for constructing synthetic genes are well-known to those of skill in the art. See, e.g., Dennis, M. S., Carter, P. and Lazarus, R. A., Proteins: Struct. Funct. Genet., 15:312-321 (1993).

The expression vector includes one or more control sequences capable of effecting and/or enhancing the expression of an operably linked polypeptide coding sequence. Control sequences that are suitable for expression in prokaryotes, for example, include a promoter sequence, an operator sequence, and a ribosome binding site. Control sequences for expression in eukaryotic cells include a promoter, an enhancer, and a transcription termination sequence (i.e., a polyadenylation signal).

An expression vector according to the invention can also include other sequences, such as, for example, nucleic acid sequences encoding a signal sequence or an amplifiable gene. A signal sequence can direct the secretion of a polypeptide fused thereto from a cell expressing the protein. In the expression vector, nucleic acid encoding a signal sequence is linked to a polypeptide coding sequence so as to preserve the reading frame of the polypeptide coding sequence. The inclusion in a vector of a gene complementing an auxotrophic deficiency in the chosen host cell allows for the selection of host cells transformed with the vector.

A wide variety of host cells are available for propagation and/or expression of vectors. Examples include prokaryotic cells (such as E. coli and strains of Bacillus, Pseudomonas, and other bacteria), yeast or other fungal cells (including S. cerevesiae and P. pastoris), insect cells, plant cells, and phage, as well as higher eukaryotic cells (such as human embryonic kidney cells and other mammalian cells).

Vectors expressing endogenous cardiovascular antigen can be introduced into a host cell by any convenient method, which will vary depending on the vector-host system employed. Generally, a vector is introduced into a host cell by transformation or infection (also known as “transfection”) with a virus (e.g., phage) bearing the vector. If the host cell is a prokaryotic cell (or other cell having a cell wall), convenient transformation methods include the calcium treatment method described by Cohen, et al. (1972) Proc. Natl. Acad. Sci., USA, 69:2110-14. If a prokaryotic cell is used as the host and the vector is a phagemid vector, the vector can be introduced into the host cell by transfection. Yeast cells can be transformed using polyethylene glycol, for example, as taught by Hinnen (1978) Proc. Natl. Acad. Sci, USA, 75:1929-33. Mammalian cells are conveniently transformed using the calcium phosphate precipitation method described by Graham, et al. (1978) Virology, 52:546 and by Gorman, et al. (1990) DNA and Prot. Eng. Tech., 2:3-10. However, other known methods for introducing DNA into host cells, such as nuclear injection, electroporation, protoplast fusion, and other means also are acceptable for use in the invention.

Expression of endogenous antigen from a transformed host cell entails culturing the host cell under conditions suitable for cell growth and expression and recovering the expressed polypeptides from a cell lysate or, if the polypeptides are secreted, from the culture medium. In particular, the culture medium contains appropriate nutrients and growth factors for the host cell employed. The nutrients and growth factors are, in many cases, well known or can be readily determined empirically by those skilled in the art. Suitable culture conditions for mammalian host cells, for instance, are described in Mammalian Cell Culture (Mather ed., Plenum Press 1984) and in Barnes and Sato (1980) Cell 22:649.

In addition, the culture conditions should allow transcription, translation, and protein transport between cellular compartments. Factors that affect these processes are well-known and include, for example, DNA/RNA copy number;

factors that stabilize DNA; nutrients, supplements, and transcriptional inducers or repressors present in the culture medium; temperature, pH and osmolality of the culture; and cell density. The adjustment of these factors to promote expression in a particular vector-host cell system is within the level of skill in the art. Principles and practical techniques for maximizing the productivity of in vitro mammalian cell cultures, for example, can be found in Mammalian Cell Biotechnology: a Practical Approach (Butler ed., IRL Press (1991).

Any of a number of well-known techniques for large- or small-scale production of proteins can be employed in producing the polypeptides of the invention. These include, but are not limited to, the use of a shaken flask, a fluidized bed bioreactor, a roller bottle culture system, and a stirred tank bioreactor system. Cell culture can be carried out in a batch, fed-batch, or continuous mode.

Methods for recovery of recombinant proteins produced as described above are well-known and vary depending on the expression system employed. A polypeptide including a signal sequence can be recovered from the culture medium or the periplasm. Polypeptides can also be expressed intracellularly and recovered from cell lysates.

The expressed polypeptides can be purified from culture medium or a cell lysate by any method capable of separating the polypeptide from one or more components of the host cell or culture medium. Typically, the polypeptide is separated from host cell and/or culture medium components that would interfere with the intended use of the polypeptide. As a first step, the culture medium or cell lysate is usually centrifuged or filtered to remove cellular debris. The supernatant is then typically concentrated or diluted to a desired volume or diafiltered into a suitable buffer to condition the preparation for further purification.

The polypeptide can then be further purified using well-known techniques. The technique chosen will vary depending on the properties of the expressed polypeptide. If, for example, the polypeptide is expressed as a fusion protein containing an epitope tag or other affinity domain, purification typically includes the use of an affinity column containing the cognate binding partner. For instance, polypeptides fused with green fluorescent protein, hemagglutinin, or FLAG epitope tags or with hexahistidine or similar metal affinity tags can be purified by fractionation on an affinity column.

Antibodies

Antibodies useful in the immunoassay methods of the invention include polyclonal and monoclonal antibodies. Polyclonal antibodies are raised by injecting (e.g., subcutaneous or intramuscular injection) an immunogen into a suitable non-human mammal (e.g., a mouse or a rabbit). Generally, the immunogen should induce production of high titers of antibody with relatively high affinity for the target antigen.

If desired, the endogenous antigen (i.e., analyte of interest) may be conjugated to a carrier protein by conjugation techniques that are well known in the art. Commonly used carriers include keyhole limpet hemocyanin (KLH), thyroglobulin, bovine serum albumin (BSA), and tetanus toxoid. The conjugate is then used to immunize the animal.

The antibodies are then obtained from blood samples taken from the animal. The techniques used to produce polyclonal antibodies are extensively described in the literature (see, e.g., Methods of Enzymology, “Production of Antisera With Small Doses of Immunogen: Multiple Intradermal Injections,” Langone, et al. eds. (Acad. Press, 1981)). Polyclonal antibodies produced by the animals can be further purified, for example, by binding to and elution from a matrix to which the target antigen is bound. Those of skill in the art will know of various techniques common in the immunology arts for purification and/or concentration of polyclonal, as well as monoclonal, antibodies see, for example, Coligan, et al. (1991) Unit 9, Current Protocols in Immunology, Wiley Interscience.

For many applications, monoclonal antibodies (mAbs) are preferred. The general method used for production of hybridomas secreting mAbs is well known (Kohler and Milstein (1975) Nature, 256:495). Briefly, as described by Kohler and Milstein, the technique entailed isolating lymphocytes from regional draining lymph nodes of five separate cancer patients with either melanoma, teratocarcinoma or cancer of the cervix, glioma or lung, (where samples were obtained from surgical specimens), pooling the cells, and fusing the cells with SHFP-1. Hybridomas were screened for production of antibody that bound to cancer cell lines. Confirmation of specificity among mAbs can be accomplished using routine screening techniques (such as the enzyme-linked immunosorbent assay, or “ELISA”) to determine the elementary reaction pattern of the mAb of interest.

As used herein, the term “antibody” encompasses antigen-binding antibody fragments, e.g., single chain antibodies (scFv or others), which can be produced/selected using phage display or yeast display technology. The ability to express antibody fragments on the surface of viruses that infect bacteria (bacteriophage or phage) makes it possible to isolate a single binding antibody fragment, e.g., from a library of greater than 1010 nonbinding clones. To express antibody fragments on the surface of phage (phage display), an antibody fragment gene is inserted into the gene encoding a phage surface protein (e.g., pIII) and the antibody fragment-pIII fusion protein is displayed on the phage surface (McCafferty et al. (1990) Nature, 348: 552-554; Hoogenboom et al. (1991) Nucleic Acids Res. 19: 4133-4137).

Since the antibody fragments on the surface of the phage are functional, phage-bearing antigen-binding antibody fragments can be separated from non-binding phage by antigen affinity chromatography (McCafferty et al. (1990) Nature, 348: 552-554). Depending on the affinity of the antibody fragment, enrichment factors of 20-fold-1,000,000-fold are obtained for a single round of affinity selection. By infecting bacteria with the eluted phage, however, more phage can be grown and subjected to another round of selection. In this way, an enrichment of 1000-fold in one round can become 1,000,000-fold in two rounds of selection (McCafferty et al. (1990) Nature, 348: 552-554). Thus, even when enrichments are low (Marks et al. (1991) J. Mol. Biol. 222: 581-597), multiple rounds of affinity selection can lead to the isolation of rare phage. Since selection of the phage antibody library on antigen results in enrichment, the majority of clones bind antigen after as few as three to four rounds of selection. Thus only a relatively small number of clones (several hundred) need to be analyzed for binding to antigen.

Human antibodies can be produced without prior immunization by displaying very large and diverse V-gene repertoires on phage (Marks et al. (1991) J. Mol. Biol. 222: 581-597). In one embodiment, natural VH and VL repertoires present in human peripheral blood lymphocytes are isolated from unimmunized donors by PCR. The V-gene repertoires can be spliced together at random using PCR to create a scFv gene repertoire which can be cloned into a phage vector to create a library of 30 million phage antibodies (Id.). From a single “naïve” phage antibody library, binding antibody fragments have been isolated against more than 17 different antigens, including haptens, polysaccharides, and proteins (Marks et al. (1991) J. Mol. Biol. 222: 581-597; Marks et al. (1993). Bio/Technology. 10: 779-783; Griffiths et al. (1993) EMBO J. 12: 725-734; Clackson et al. (1991) Nature. 352: 624-628). Antibodies have been produced against self proteins, including human thyroglobulin, immunoglobulin, tumor necrosis factor, and CEA (Griffiths et al. (1993) EMBO J. 12: 725-734). The antibody fragments are highly specific for the antigen used for selection and have affinities in the 1 nM to 100 nM range (Marks et al. (1991) J. Mol. Biol. 222: 581-597; Griffiths et al. (1993) EMBO J. 12: 725-734). Larger phage antibody libraries result in the isolation of more antibodies of higher binding affinity to a greater proportion of antigens.

As those of skill in the art readily appreciate, antibodies can be prepared by any of a number of commercial services (e.g., Berkeley Antibody Laboratories, Bethyl Laboratories, Anawa, Eurogenetec, etc.).

Solid Phase

For embodiments of the invention that employ a solid phase as a support for the capture agent, the solid phase can be any suitable material with sufficient surface affinity to bind a capture agent. Useful solid supports include: natural polymeric carbohydrates and their synthetically modified, crosslinked, or substituted derivatives, such as agar, agarose, cross-linked alginic acid, substituted and cross-linked guar gums, cellulose esters, especially with nitric acid and carboxylic acids, mixed cellulose esters, and cellulose ethers; natural polymers containing nitrogen, such as proteins and derivatives, including cross-linked or modified gelatins; natural hydrocarbon polymers, such as latex and rubber; synthetic polymers, such as vinyl polymers, including polyethylene, polypropylene, polystyrene, polyvinylchloride, polyvinylacetate and its partially hydrolyzed derivatives, polyacrylamides, polymethacrylates, copolymers and terpolymers of the above polycondensates, such as polyesters, polyamides, and other polymers, such as polyurethanes or polyepoxides; inorganic materials such as sulfates or carbonates of alkaline earth metals and magnesium, including barium sulfate, calcium sulfate, calcium carbonate, silicates of alkali and alkaline earth metals, aluminum and magnesium; and aluminum or silicon oxides or hydrates, such as clays, alumina, talc, kaolin, zeolite, silica gel, or glass (these materials may be used as filters with the above polymeric materials); and mixtures or copolymers of the above classes, such as graft copolymers obtained by initializing polymerization of synthetic polymers on a pre-existing natural polymer. All of these materials may be used in suitable shapes, such as films, sheets, tubes, particulates, or plates, or they may be coated onto, bonded, or laminated to appropriate inert carriers, such as paper, glass, plastic films, fabrics, or the like.

Nitrocellulose has excellent absorption and adsorption qualities for a wide variety of reagents including monoclonal antibodies. Nylon also possesses similar characteristics and also is suitable.

Preferred solid phase materials for flow-through assay devices include filter paper such as a porous fiberglass material or other fiber matrix materials. The thickness of such material is not critical and will be a matter of choice, largely based upon the properties of the biological sample or analyte being assayed, such as the fluidity of the biological sample.

Alternatively, the solid phase can constitute microparticles. Microparticles useful in the invention can be selected by one skilled in the art from any suitable type of particulate material and include those composed of polystyrene, polymethylacrylate, polypropylene, latex, polytetrafluoroethylene, polyacrylonitrile, polycarbonate, or similar materials. Further, the microparticles can be magnetic or paramagnetic microparticles, so as to facilitate manipulation of the microparticle within a magnetic field.

Microparticles can be suspended in the mixture of soluble reagents and biological sample or can be retained and immobilized by a support material. In the latter case, the microparticles on or in the support material are not capable of substantial movement to positions elsewhere within the support material. Alternatively, the microparticles can be separated from suspension in the mixture of soluble reagents and biological sample by sedimentation or centrifugation. When the microparticles are magnetic or paramagnetic the microparticles can be separated from suspension in the mixture of soluble reagents and biological sample by a magnetic field.

The methods of the present invention can be adapted for use in systems that utilize microparticle technology including automated and semi-automated systems wherein the solid phase comprises a microparticle. Such systems include those described in pending U.S. application Ser. No. 425,651 and U.S. Pat. No. 5,089,424, which correspond to published EPO App. Nos. EP 0 425 633 and EP 0 424 634, respectively, and U.S. Pat. No. 5,006,309.

In particular embodiments, the solid phase includes one or more electrodes. Capture agent(s) can be affixed, directly or indirectly, to the electrode(s). In one embodiment, for example, capture agents can be affixed to magnetic or paramagnetic microparticles, which are then positioned in the vicinity of the electrode surface using a magnet. Systems in which one or more electrodes serve as the solid phase are useful where detection is based on electrochemical interactions. Exemplary systems of this type are described, for example, in U.S. Pat. No. 6,887,714 (issued May 3, 2005). The basic method is described further below with respect to electrochemical detection.

The capture agent can be attached to the solid phase by adsorption, where it is retained by hydrophobic forces. Alternatively, the surface of the solid phase can be activated by chemical processes that cause covalent linkage of the capture agent to the support.

To change or enhance the intrinsic charge of the solid phase, a charged substance can be coated directly onto the solid phase. Ion capture procedures for immobilizing an immobilizable reaction complex with a negatively charged polymer, described in U.S. application Ser. No. 150,278, corresponding to EP Publication No. 0326100, and U.S. application Ser. No. 375,029 (EP Publication No. 0406473), can be employed according to the present invention to affect a fast solution-phase immunochemical reaction. In these procedures, an immobilizable immune complex is separated from the rest of the reaction mixture by ionic interactions between the negatively charged polyanion/immune complex and the previously treated, positively charged matrix and detected by using any of a number of signal-generating systems, including, e.g., chemiluminescent systems, as described in U.S. application Ser. No. 921,979, corresponding to EPO Publication No. 0 273,115.

If the solid phase is silicon or glass, the surface must generally be activated prior to attaching the specific binding partner. Activated silane compounds such as triethoxy amino propyl silane (available from Sigma Chemical Co., St. Louis, Mo.), triethoxy vinyl silane (Aldrich Chemical Co., Milwaukee, Wis.), and (3-mercapto-propyl)-trimethoxy silane (Sigma Chemical Co., St. Louis, Mo.) can be used to introduce reactive groups such as amino-, vinyl, and thiol, respectively. Such activated surfaces can be used to link the capture directly (in the cases of amino or thiol), or the activated surface can be further reacted with linkers such as glutaraldehyde, bis (succinimidyl) suberate, SPPD 9 succinimidyl 3-[2-pyridyldithio]propionate), SMCC (succinimidyl-4-[Nmaleimidomethyl]cyclohexane-1-carboxylate), SIAB (succinimidyl[4iodoacetyl]aminobenzoate), and SMPB (succinimidyl 4-[1maleimidophenyl]butyrate) to separate the capture agent from the surface. Vinyl groups can be oxidized to provide a means for covalent attachment. Vinyl groups can also be used as an anchor for the polymerization of various polymers such as poly-acrylic acid, which can provide multiple attachment points for specific capture agents. Amino groups can be reacted with oxidized dextrans of various molecular weights to provide hydrophilic linkers of different size and capacity. Examples of oxidizable dextrans include Dextran T-40 (molecular weight 40,000 daltons), Dextran T-110 (molecular weight 110,000 daltons), Dextran T-500 (molecular weight 500,000 daltons), Dextran T-2M (molecular weight 2,000,000 daltons) (all of which are available from Pharmacia, Piscataway, N.J.), or Ficoll (molecular weight 70,000 daltons; available from Sigma Chemical Co., St. Louis, Mo.). Additionally, polyelectrolyte interactions can be used to immobilize a specific capture agent on a solid phase using techniques and chemistries described U.S. application Ser. No. 150,278, filed Jan. 29, 1988, and U.S. application Ser. No. 375,029, filed Jul. 7, 1989, each of which is incorporated herein by reference.

Other considerations affecting the choice of solid phase include the ability to minimize non-specific binding of labeled entities and compatibility with the labeling system employed. For, example, solid phases used with fluorescent labels should have sufficiently low background fluorescence to allow signal detection. Following attachment of a specific capture agent, the surface of the solid support may be further treated with materials such as serum, proteins, or other blocking agents to minimize non-specific binding.

Labeling Systems

As discussed above, many immunoassays according to the invention employ a labeled detection agent, such as a labeled antibody or a labeled antigen.

Detectable labels suitable for use in the detection agents of the present invention include any composition detectable by spectroscopic, photochemical, biochemical, immunochemical, electrical, optical, or chemical means. Useful labels in the present invention include magnetic beads (e.g., Dynabeads™), fluorescent dyes (e.g., fluorescein, Texas Red, rhodamine, green fluorescent protein, and the like, see, e.g., Molecular Probes, Eugene, Oreg., USA), chemiluminescent compounds such as acridinium (e.g., acridinium-9-carboxamide), phenanthridinium, dioxetanes, luminol and the like, radiolabels (e.g., 3H, 125I, 35S, 14C, or 32P), catalysts such as enzymes (e.g., horseradish peroxidase, alkaline phosphatase, beta-galactosidase and others commonly used in an ELISA), and colorimetric labels such as colloidal gold (e.g., gold particles in the 40-80 nm diameter size range scatter green light with high efficiency) or colored glass or plastic (e.g., polystyrene, polypropylene, latex, etc.) beads. Patents teaching the use of such labels include U.S. Pat. Nos. 3,817,837; 3,850,752; 3,939,350; 3,996,345; 4,277,437; 4,275,149; and 4,366,241.

The label can be attached to the detection agent prior to, or during, or after contact with the biological sample. So-called “direct labels” are detectable labels that are directly attached to or incorporated into detection agents prior to use in the assay. Direct labels can be attached to or incorporated into detection agents by any of a number of means well known to those of skill in the art.

In contrast, so-called “indirect labels” typically bind to the detection agent at some point during the assay. Often, the indirect label binds to a moiety that is attached to or incorporated into the detection agent prior to use. Thus, for example, an antibody used as a detection agent (a “detection antibody”) can be biotinylated before use in an assay. During the assay, an avidin-conjugated fluorophore can bind the biotin-bearing detection agent, to provide a label that is easily detected. In another example of indirect labeling, polypeptides capable of specifically binding immunoglobulin constant regions, such as polypeptide A or polypeptide G, can also be used as labels for detection antibodies. These polypeptides are normal constituents of the cell walls of streptococcal bacteria. They exhibit a strong non-immunogenic reactivity with immunoglobulin constant regions from a variety of species (see, generally Kronval, et al. (1973) J. Immunol., 111: 1401-1406, and Akerstrom (1985) J. Immunol., 135: 2589-2542). Such polypeptides can thus be labeled and added to the assay mixture, where they will bind to the detection antibody, as well as to the species-specific antibody, labeling both and providing a composite signal attributable to analyte and autoantibody present in the biological sample.

Some labels useful in the invention may require the use of an indicator reagent to produce a detectable signal. In an ELISA, for example, an enzyme label (e.g., beta-galactosidase) will require the addition of a substrate (e.g., X-gal) to produce a detectable signal.

Exemplary Formats

Electrochemical Detection Systems

The present invention is for example applicable (e.g., adaptable) to the jointly owned commercial Abbott Point of Care (i-STAT®) electrochemical immunoassay system which performs sandwich immunoassays for several cardiac markers, including TnI, CKMB and BNP. Immunosensors and ways of operating them in single-use test devices are described in jointly owned Publication Nos. US 20030170881, US 20040018577, US 20050054078, and US 20060160164, each of which is incorporated herein by reference. Additional background on the manufacture of electrochemical and other types of immunosensors is found in jointly owned U.S. Pat. No. 5,063,081 which is also incorporated by reference.

Multiplex Formats (Exemplary Panel)

In particular embodiments, useful, for example, for simultaneously assaying multiple analytes in one biological sample, the solid phase can include a plurality of different capture agents, including one that captures endogenous antigen or analyte of interest (e.g., homocysteine). Thus, for example, the solid phase can have affixed thereon a plurality of antibodies, wherein each is intended to test for the presence of different analytes (e.g., homocysteine and endogenous analytes) in the biological sample. In an exemplary embodiment, the solid phase can consist of a plurality of different regions on a surface, wherein each region has a particular antibody affixed therein.

Multiplex formats can, but need not, employ a plurality of labels, wherein each label is used for the detection of a particular antigen. For example, multiple, different analytes can be detected without using a plurality of labels where a plurality of capture agents, such as antibodies having different specificites, are affixed to the solid phase at different known locations. Because the specificity of the capture agent at each location is known, the detection of a signal at a particular location can be associated with the presence of antigen bound at that location. Examples of this format include microfluidic devices and capillary arrays, containing different capture agents at different locations along a channel or capillary, respectively, and microarrays, which typically contain different capture agents arranged in a matrix of spots (“target elements”) on a surface of a solid support. In particular embodiments, each different capture agent can be affixed to a different electrode, which can, for example, be formed on a surface of a solid support, in a channel of a microfluidic device, or in a capillary.

Automated Instrumentation

Optionally the immunoassays as described herein can be used in kits for commercial platform immunoassays (e.g., homocysteine blood screening assays on Abbott's Prism®, AxSYM®, ARCHITECT® and/or EIA (Bead) platforms, as well as in other commercial and/or in vitro diagnostic assays.

Test Kits

The invention also provides test kits for assaying biological samples for analytes such as homocysteine and other endogenous antigens. Test kits according to the invention include one or more reagents useful for practicing one or more immunoassays according to the invention. A test kit generally includes a package with one or more containers holding the reagents, as one or more separate compositions or, optionally, as admixture where the compatibility of the reagents will allow. The test kit can also include other material(s), which may be desirable from a user standpoint, such as a buffer(s), a diluent(s), a standard(s), and/or any other material useful in biological sample processing, washing, or conducting any other step of the assay.

In certain embodiments, a test kit includes a polyanion, wherein the polyanion is employed in the reaction of analyte antigen with antibody. If desired, this component can be included in the test kit in multiple concentrations, and/or by provision of a variety of different types of polyanions (including mixtures).

Kits according to the invention can include a solid phase and a capture agent affixed to the solid phase, wherein the capture agent is an antibody specific for the analyte being assessed in the biological sample. Where such kits are to be employed for conducting sandwich immunoassays, the kits can additionally include a labeled detection agent.

In certain embodiments, the test kit includes at least one direct label, such as acridinium-9-carboxamide. Test kits according to the invention can also include at least one indirect label. If the label employed generally requires an indicator reagent to produce a detectable signal, the test kit preferably includes one or more suitable indicator reagents.

In exemplary embodiments, the solid phase includes one or more microparticles or electrodes. Test kits designed for multiplex assays conveniently contain one or more solid phases including a plurality of antibodies that are specific for a plurality of different analytes of interest (e.g., homocysteine or endogenous antigens). Thus, for example, a test kit designed for multiplex electrochemical immunoassays can contain a solid phase including a plurality of electrodes, with each electrode bearing a different antibody.

Test kits according to the invention preferably include instructions for carrying out one or more of the immunoassays of the invention. Instructions included in kits of the invention can be affixed to packaging material or can be included as a package insert. While the instructions are typically written or printed materials they are not limited to such. Any medium capable of storing such instructions and communicating them to an end user is contemplated by this invention. Such media include, but are not limited to, electronic storage media (e.g., magnetic discs, tapes, cartridges, chips), optical media (e.g., CD ROM), and the like. As used herein, the term “instructions” can include the address of an internet site that provides the instructions.

The invention will be better understood through the following Examples illustrating its use and efficacy. The following Examples are offered to illustrate, but not to limit, the scope and essential features of the present invention.

### EXAMPLES

The following reagents and methods were employed for measurement of homocysteine using a fully automated chemiluminescence measuring apparatus

Reagents

An anti-S-adenosylhomocysteine mouse monoclonal antibody (procured from Abbott Laboratories, U.S.A.) was bonded onto a magnetic fine particles modified by the addition of carboxyl group (procured from Abbott Laboratories, U.S.A.) using EDC (N-ethyl-N′-(3-dimethylaminopropyl) carbodiimide hydrochloride (manufactured by Sigma Aldrich)) to give fine particles where the antibody was made into a solid phase. The antibody in a solid phase was added to a BisTris buffer solution containing Tween 20 (manufactured by Kanto Kagaku), EDTA (sodium ethylenediaminetetraacetate) and sodium chloride to prepare a solution of fine particles of the antibody in a solid phase.

S-adenosylcysteine labeled with acridinium derivative (procured from Abbott Laboratories, U.S.A.) was added to an MES buffer solution containing Triton X 100 (manufactured by Sigma Aldrich) to prepare a tracer solution.

S-adenosylhomocysteine hydrolase (procured from Axis Shield, United Kingdom) was added to a buffer solution containing 30% (by volume) of glycerol to prepare an enzyme solution.

DTT and adenosine were added to an aqueous solution of citric acid to prepare a reducing agent solution.

Method

The following operations and measurement were carried out using an Architect® fully automated immunoassay analyzer (manufactured by Abbott Japan Co., Ltd.). A sample (18 μL) was mixed with 79 μL of the enzyme solution, 50 μL of the fine particles solution of antibody in a solid phase, and 10 μL of the reducing agent solution, and the first reaction was started. In this mixed solution, the following reactions were generated: (1) a bonded product of homocysteine in the sample was liberated to a free homocysteine; (2) the liberated homocysteine was converted to S-adenosylhomocysteine; and (3) the converted S-adenosylhomocysteine was bonded to the particles of antibody in a solid phase.

After 21 minutes, the tracer solution (50 μL) was further mixed therewith and the reaction was continued for 4 minutes. As a result of this reaction, the aforementioned reaction (3), and a competitive reaction to the fine particles of antibody in a solid phase among the tracers resulted and, depending upon the concentration of homocysteine in the sample, the tracers were competitively bonded to the fine particles of antibody in a solid phase. Thus, if the homocysteine concentration in the sample was low, many tracers were bonded to the fine particles of antibody in a solid phase. In contrast, when the homocysteine concentration in the sample was high, small numbers of tracers were bonded to the fine particles of antibody in a solid phase.

Then, after washing with a washing liquid which was exclusive for this instrument, an emission signal was observed using an emission trigger reagent (also was exclusive for this instrument). A standard curve was prepared by a logistic 4 para method using an Abbott AxSYM® homocysteine calibrator (manufactured by Abbott Japan Co., Ltd.) as a reference solution, and the homocysteine concentration was calculated based on the signal obtained from the sample, whereupon the concentration of the homocysteine in the sample was determined.

### Example 1

The concentration of homocysteine contained in the sample was determined by prior art methods including a diluting operation using a commercially-available AxSYM® Homocysteine Assay Reagent and AxSYM® Analyzer (both manufactured by Abbott Japan Co., Ltd.). This measuring method is a fluorescence polarization immunoassay where a fluorescent substance is utilized in a labeled substance and, in order to eliminate the influence of an inhibiting substance, the sample was diluted to an extent of about 300-fold upon a competitive reaction.

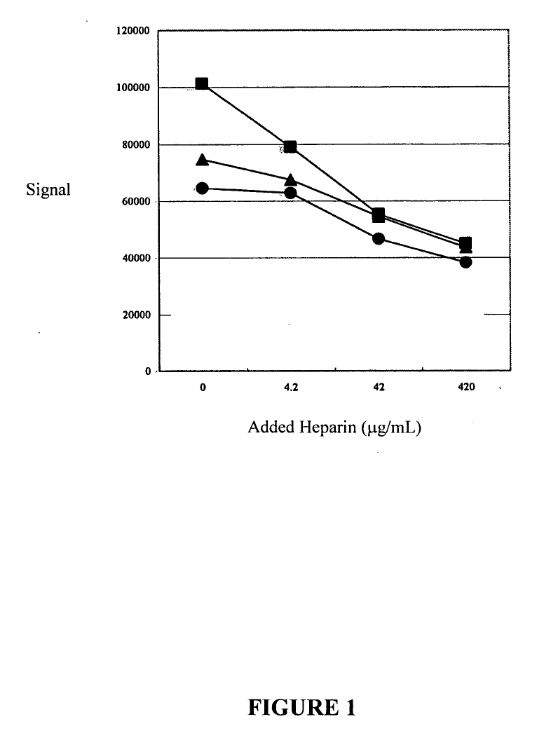

In order to confirm the influence of the inhibiting substance on the measuring system and the effect of polyanion, the concentration of homocysteine was measured similarly for undiluted serum, and also for samples where 4.2, 42 and 420 μg/mL of heparin was added to the serum. The results of these tests are shown in FIG. 1.

It was confirmed that, in the undiluted sample, serum (▪) in which the concentration of homocysteine was able to be precisely measured, and serum (●) in which the signal intensity was detected to be a bit low (whereby the apparent concentration of homocysteine was measured high).

In those two kinds of undiluted serums, although a big difference was noted in signal intensity, in neither case was heparin was added. It was found that, as a result of addition of heparin, the difference between the assay results became small, and that when 42 μg/mL of heparin was added, there was almost no difference between them in terms of assay results.

From the above, it appears that a heparin-like anionic substances derived from a living organism was contained in the latter serum (●), and that those substances inhibited the present measuring system. By contrast, in the former serum (▪), apparently no substantial amount of inhibiting substance was present and as consequence, the influence of the added heparin was stronger.

Plasma (▾) in blood collected with heparin (e.g., heparinized tubes) was similarly investigated and experimental results were obtained similar to those for the serum where the homocysteine concentration was apparently highly measured (●). Because of that, it is also strongly suggested that a heparin-like polyanionic substance derived from a living organism was contained in the serum where homocysteine concentration was apparently highly measured.

From the aforementioned results, it was confirmed that, when a sufficient amount of polyanion is added to the sample, it is possible to eliminate the variation in measured data caused by the amount of inhibiting substances contained in each sample.

In order to confirm whether a rheumatoid factor participates as an inhibiting substance in a measuring system for homocysteine, the concentration of rheumatoid factor was quantified for the sample used in the Examples. As a result of the measurement for six samples where homocysteine was able to be precisely measured and for four samples where it was measured in apparently high concentrations, only one among the samples where homocysteine was able to be precisely measured contained the rheumatoid factor in an amount of more than the standard value while all other samples were within a range of normal value. This suggests that a rheumatoid factor does not participate as a reaction inhibitor in an immunoassay of homocysteine.

### Example 2

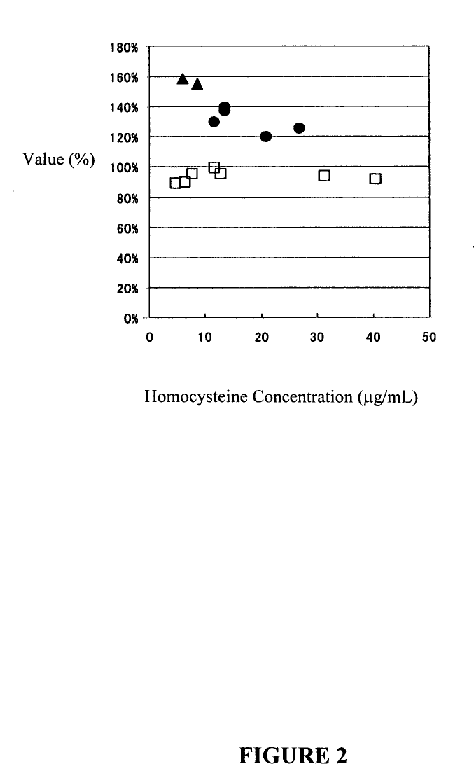

Twelve kinds of serum and two kinds of plasma in blood collected with heparin were subjected to measurement of homocysteine concentration using a fully automated chemiluminescent measuring apparatus, and each concentration was compared with the homocysteine concentration (known value) determined by an AxSYM® Analyzer in the same manner as in Example 1. The results of these experiments are shown in FIG. 2.

As can be seen in FIG. 2, out of the twelve kinds of serum, the concentrations of seven of the serums (□) were no different from the actual homocysteine concentration, whereas for five kinds thereof (●), higher measured concentrations than the actual concentration were obtained. In the plasma in blood collected with heparin, both samples showed higher measured concentrations than the actual concentration (▾).

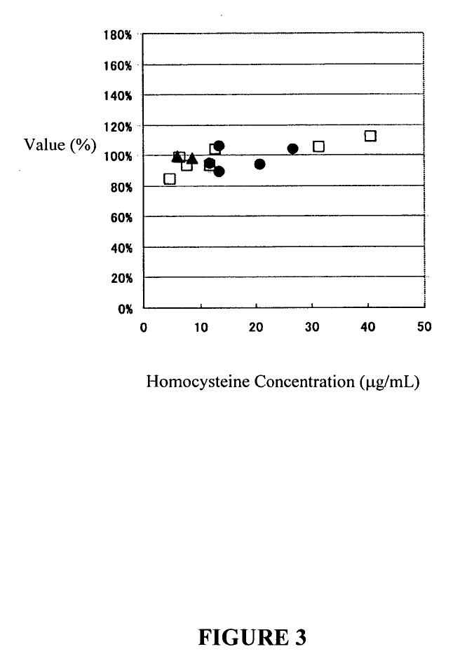

An immunoreaction also was conducted using the same serum and plasma samples in the presence of 42 μg/mL of heparin. The measured result are shown in FIG. 3.

As can be seen from FIG. 3, in both cases of the plasma samples, and the five samples where a higher measured value was obtained than the actual one, with addition of heparin the concentrations were then able to be precisely measured.

The above experimental result confirm that addition of a polyanion such as heparin is able to eliminate the influence of an inhibiting substance, and also that the homocysteine concentration was able to be precisely measured in the presence of polyanion.

### Example 3

It also was investigated whether the addition of polyanion other than heparin was similarly able to eliminate the influence of an inhibiting substance.

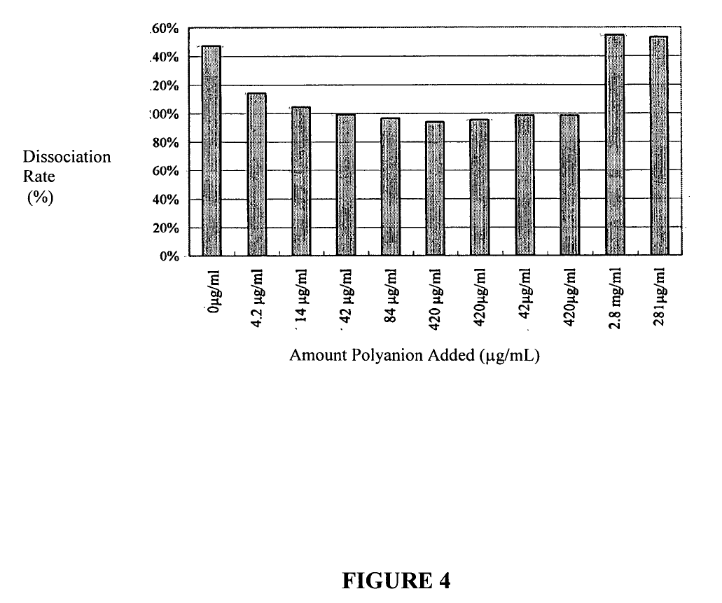

FIG. 4 shows rates of dissociation in the higher sample group and the normal sample group at each of the concentrations of dextran sulfate, polyacrylic acid, gelatin and γ-globulin. Each 7 and 8 samples were used as the higher and the normal sample groups, respectively.

In order to eliminate the dispersion in the measured values among the samples by the inhibiting substance and to appropriately measure the homocysteine concentration, it is necessary that such values are within about 100%±10%.

It is noted from FIG. 4 that, when not less than 4.2 μg/mL of heparin is present in the first reaction, the influence of inhibition by the sample was able to be effectively avoided. Further, all of the investigated three kinds of polyanions (heparin, dextran sulfate, and polyacrylic acid) showed a significant effect whereas in the case of gelatin or γ-globulin which are not polyanions, such an effect was not achieved. Accordingly, the effect of eliminating the influence of the inhibiting substance was found to be common in polyanions.

Typical heparin has one anion per a molecular weight of 150 whereas in some polyanions, there are molecules in which the anions are more densely present.

For example, polyacrylic acid has one anion per a molecular weight of 71. When the presence in the polyanion molecule of anions being densely present is further taken into consideration, it is expected that an ability to eliminate or suppress the influence of the inhibiting substance is achieved when the polyanion concentration is not more than about 1 μg/mL.

The disclosure of all patents, publications, including published patent applications, and database entries referenced in this specification are specifically incorporated by reference in their entirety to the same extent as if each such individual patent, publication, and database entry were specifically and individually indicated to be incorporated by reference.

Although the invention has been described with reference to certain specific embodiments, various modifications thereof will be apparent to those skilled in the art without departing from the spirit and scope of the invention. All such modifications as would be apparent to one skilled in the art are intended to be included within the scope of the following claims.

What is claimed is:

1. In an improvement of a homocysteine immunoassay of a biological sample, characterized in that the sample is reacted with antibody in the presence of an extrinsic polyanion.

2. The homocysteine immunoassay according to claim 1, wherein the polyanion is selected from the group consisting of heparin, polyacrylic acid, and dextran sulfate.

3. The homocysteine immunoassay according to claim 1, wherein the polyanion concentration ranges from about 1 μg/mL to about 100 mg/mL.

4. The homocysteine immunoassay according to claim 1, wherein said immunoassay comprises a competitive immunoassay.

5. The homocysteine immunoassay according to claim 1, wherein said immunoassay is carried out in an automated measuring apparatus,

6. The homocysteine immunoassay according to claim 1, wherein said immunoassay comprises a sandwich assay.

7. A kit to be used for the homocysteine immunoassay according to claim 1, wherein said kit comprises a polyanion.

8. An immunodiagnostic reagent for the detection of homocysteine, wherein said immunodiagnostic reagent comprises antibody specific for homocysteine and polyanion.

9. The immunodiagnostic reagent according to claim 8, wherein said polyanion comprises a plurality of polyanions.

10. A kit for assaying a biological sample for homocysteine, said kit comprising the immunodiagnostic reagent of claim 8.

11. A kit for assaying a biological sample for homocysteine, said kit comprising antibody specific for homocysteine and polyanion.

12. A method for assaying a biological sample for homocysteine, wherein said method comprises:
(a) obtaining a biological sample from a subject;
(b) reacting said biological sample with antibody specific for homocysteine in the presence of polyanion;
(c) detecting the binding of homocysteine present in said sample with said antibody; and
(d) quantifying the binding as a measure of the amount of homocysteine present in said sample.

13. The method of claim 12, wherein said polyanion comprises a plurality of polyanions.

14. The method of claim 12, wherein said plurality comprises between two and five.
